(19) United States
(12) Patent Application Publication
(10) Pub. No.: US 2024/0161920 A1
(43) Pub. Date: May 16, 2024

(54) MEDICAL INFORMATION PROCESSING APPARATUS AND MEDICAL INFORMATION PROCESSING SYSTEM

(71) Applicant: Canon Medical Systems Corporation, Otawara-shi (JP)

(72) Inventor: Takanobu UCHIBORI, Otawara (JP)

(73) Assignee: Canon Medical Systems Corporation, Otawara-shi (JP)

(21) Appl. No.: 18/507,145

(22) Filed: Nov. 13, 2023

(30) Foreign Application Priority Data
Nov. 16, 2022 (JP) 2022-183638

(51) Int. Cl. G16H 40/63

(52) U.S. Cl. CPC G16H 40/63

(57) ABSTRACT

A medical information processing apparatus includes processing circuitry. The processing circuitry is configured to: acquire operator information that identifies an operator of an operation apparatus; acquire apparatus information that indicates a medical apparatus operable by the operator based on the acquired operator information; and provide the operation apparatus with the acquired apparatus information.

### CROSS-REFERENCE TO RELATED APPLICATION(S)

This application is based upon and claims the benefit of priority from Japanese Patent Application No. 2022-183638, filed Nov. 16, 2022, the entire contents of which are incorporated herein by reference.

### FIELD

Embodiments described herein relate generally to a medical information processing apparatus and a medical information processing system.

### BACKGROUND

In a case where a plurality of medical apparatuses and a plurality of operation apparatuses are connected via a common network, it is possible to use multiple medical apparatuses from one operation apparatus or to use one medical apparatus from multiple operation apparatuses. However, an operator may erroneously operate a medical apparatus that should not be operated by the operator.

### BRIEF DESCRIPTION OF THE DRAWING(S)

FIG. 1 is a block diagram showing a configuration of a medical information processing system according to a first embodiment.

FIG. 2 is a block diagram showing a configuration of a medical information processing apparatus according to the first embodiment.

FIG. 3 is a diagram showing an example of a list of medical information according to the first embodiment.

FIG. 4 is a block diagram showing a configuration of an operation apparatus according to the first embodiment.

FIG. 5 is a schematic diagram showing communication processing performed in the medical information processing system according to the first embodiment.

FIG. 6 is a sequence diagram showing communication processing performed in the medical information processing system according to the first embodiment.

FIG. 7 is a diagram showing an example of a display screen of authentication information according to the first embodiment.

FIG. 8 is a diagram showing an example of a display screen of apparatus information according to the first embodiment.

FIG. 9 is a diagram showing an example of a display screen of apparatus information according to a modification of the first embodiment.

FIG. 10 is a block diagram showing a configuration of a medical information processing apparatus according to a second embodiment.

FIG. 11 is a schematic diagram showing communication processing performed in a medical information processing system according to the second embodiment.

FIG. 12 is a sequence diagram showing communication processing performed in the medical information processing system according to the second embodiment.

FIG. 13 is a diagram showing an example of a display screen of apparatus information according to a modification of the second embodiment.

FIG. 14 is a schematic diagram showing communication processing performed in a medical information processing system according to a third embodiment.

FIG. 15 is a diagram showing an example of a display screen of authentication information according to the third embodiment.

FIG. 16 is a schematic diagram showing communication processing performed in a medical information processing system according to a fourth embodiment.

### DETAILED DESCRIPTION

In general, a medical information processing apparatus includes processing circuitry. The processing circuitry is configured to: acquire operator information that identifies an operator of an operation apparatus; acquire apparatus information that indicates a medical apparatus operable by the operator based on the acquired operator information; and provide the operation apparatus with the acquired apparatus information.

Hereinafter, embodiments of the medical information processing system will be explained in detail with reference to the accompanying drawings. In the following description, structural elements having approximately the same function and configuration will be assigned the same reference symbol, and a repetitive description will be given only where necessary.

### First Embodiment

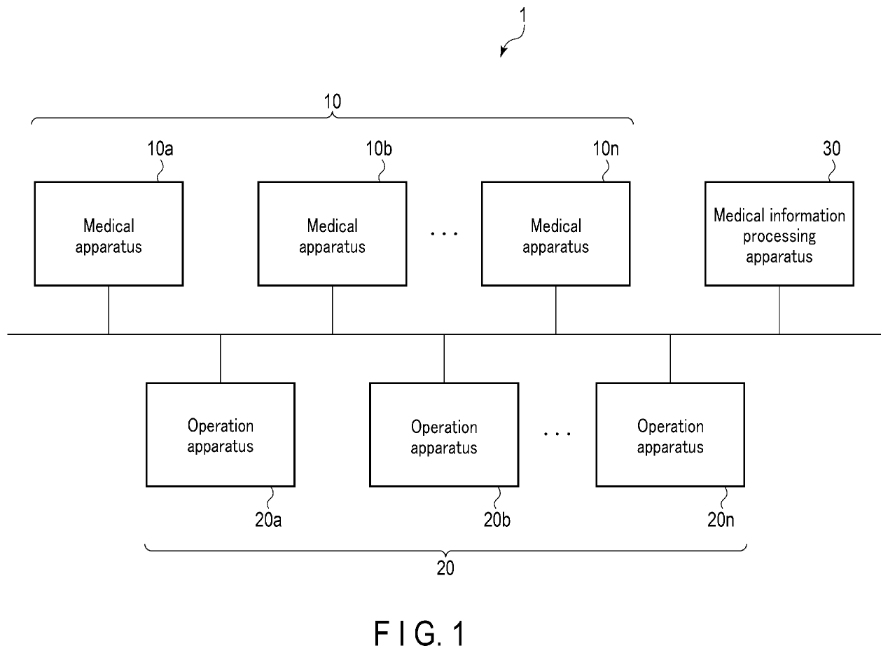

FIG. 1 is a diagram showing a configuration of a medical information processing system 1. The medical information processing system 1 is a system in which structural elements are disposed in a common network. The medical information processing system 1 includes a medical apparatus 10 (medical apparatuses 10a-10n), an operation apparatus 20 (operation apparatuses 20a-20n), and a medical information processing apparatus 30, which are communicably connected to one another via the common network. The common network is, for example, Ethernet (registered trademark), such as a local area network (LAN). The connection to the common network may be either wired or wireless. If security is ensured by a virtual private network (VPN) or the like, the line to which the apparatuses are connected is not limited to a LAN, but may be a public communication line, such as the Internet. The medical information processing system 1 includes one or more medical apparatuses 10 and one or more operation apparatuses 20.

The medical information processing system 1 achieves a high system scalability by connecting the structural elements (the medical apparatuses 10, the operation apparatuses 20, and the medical information processing apparatus 30) to the common network. For example, even on different scales, such as in units of departments of a hospital, in units of hospitals, or in units of districts, the system can be constructed in a similar design. Such a system construction can realize a medical care system architecture that supports throughout all medical scenes from in-hospital medical care to community medical care or home medical care. Furthermore, in the medical information processing system 1, since computational resources disposed in the common network can be used in common, cost-saving and space-saving can be facilitated by flexibly allocating the resources in accordance with the situation of operations. Moreover, the medical information processing system 1 has a flexible physical configuration (flexible deployment); that is, the structural elements may be disposed anywhere on the common network.

Thus, in a case where a plurality of medical apparatuses 10 and a plurality of operation apparatuses 20 are connected via the common network, it is possible to use the medical apparatuses 10 from one operation apparatus 20 or to use one medical apparatus 10 from the operation apparatuses 20. However, a medical apparatus 10 that should not be operated by an operator of an operation apparatus 20 may be erroneously operated.

The medical information processing system 1 provides the operation apparatus 20, from the medical information processing apparatus 30, with medical information including the medical apparatuses 10 operable by the operator of the operation apparatus 20. The medical information processing system 1 prevents a medical apparatus 10 that should not be operated by the operator of the operation apparatus 20 from being erroneously operated.

The medical apparatus 10 performs communications with the operation apparatus 20 or the medical information processing apparatus 30. The medical apparatus 10 is operated by the operation apparatus 20 via the common network. Examples of the medical apparatus 10 include, for example, a medical image diagnostic apparatus, a radiotherapy machine, a surgery device, and the like. Examples of the medical image diagnostic apparatus include, for example, an X-ray computed tomography apparatus (CT apparatus), a magnetic resonance imaging apparatus (MRI apparatus), an ultrasonic diagnostic apparatus, an X-ray diagnostic apparatus, a positron emission tomography apparatus (PET apparatus), and the like. The medical apparatuses 10a-10n may be disposed in the same room or different rooms in the same medical facility, or may be disposed in different medical facilities.

The medical information processing apparatus 30 performs communications with the medical apparatus 10 or the operation apparatus 20 via the common network. The medical information processing apparatus 30 comprises, for example, a server. The medical information processing apparatus 30 performs communications with the operation apparatus 20 and provides the operation apparatus 20 with a list of medical information including the medical apparatuses 10 operable by the operator of the operation apparatus 20.

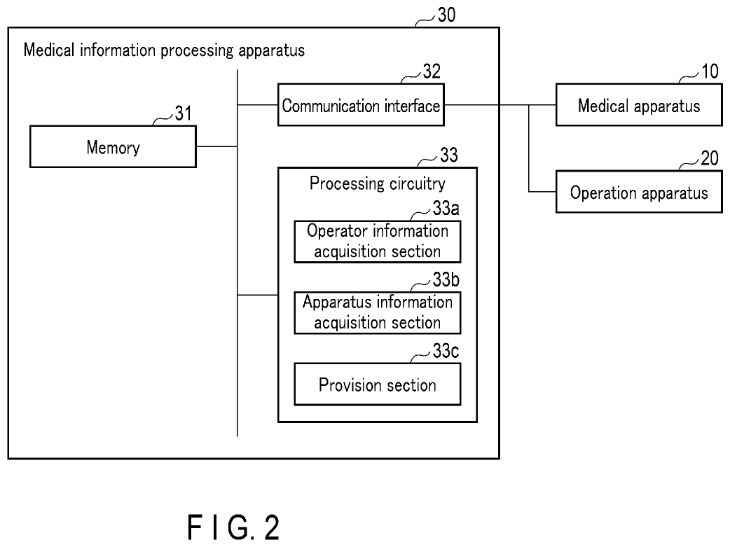

As shown in FIG. 2, the medical information processing apparatus 30 comprises a memory 31, a communication interface 32, and processing circuitry 33.

The communication interface 32 is realized by, for example, a communication apparatus, such as a network card, a network adapter, a network interface controller (NIC), or the like. The communication interface 32 performs communications with the medical apparatuses 10 and the operation apparatuses 20 via the network.

The memory 31 is realized by, for example, a semiconductor memory device such as a random access memory (RAM) or a flash memory, or a hard disk, an optical disk, etc. The memory 31 stores various data and programs necessary for processing performed by the processing circuitry 33 and various data obtained through processing performed by the processing circuitry 33. The memory 31 is an example of a storage.

The memory 31 also stores a list of medical information in which an operator is associated with a medical apparatus operable by the operator. The list of medical information is managed for each medical order. Examples of medical orders include an examination order, a medical treatment order, an image interpretation order, a clinical examination order, and the like. The list of medical information is periodically acquired via the network from, for example, a hospital information system, an electronic health record system, or the like. The list of medical information indicates the medical apparatuses 10 operable by the operator. The medical apparatuses operable by the operator refers to medical apparatuses for which the operator is given authority for operation.

The medical information may be generated by the medical information processing apparatus 30. For example, the medical information processing apparatus 30 may assign an operator to a medical apparatus included in the medical order, based on schedules of operators managed by a schedule management apparatus and medical orders generated by a medical order apparatus, to generate medical information. If the schedule management apparatus manages skills of the operators in addition to the schedules of the operators, the medical information processing apparatus 30 may assign an appropriate operator to the medical apparatus 10 included in the medical order, to generate medical information. The list of medical information generated by the medical information processing apparatus 30 is stored in the memory 31.

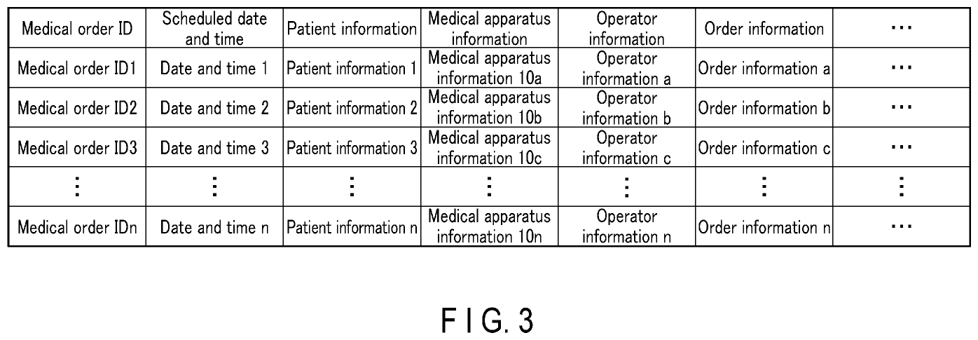

Examples of the medical information stored in the memory 31 include, for example, a medical order ID, medical apparatus information, operator information, a scheduled date and time, patient information, order information, and the like, as shown in FIG. 3. The medical order ID is information that unambiguously identifies a medical order. The medical apparatus information is information that identifies the medical apparatus 10, and includes an apparatus ID that unambiguously identifies the medical apparatus 10, the type of the medical apparatus 10, the name of the medical apparatus 10, and the like. The medical apparatus information includes information on one or more medical apparatuses 10 depending on the contents of the medical orders. The medical apparatus information includes connection information (link information) to connect to the medical apparatus 10, such as a Uniform Resource Locator (URL), an Internet Protocol (IP) address of the medical apparatus 10, or the like.

The operator information includes a user ID that unambiguously identifies an operator, a password, and an operator name. The operator information includes information on one or a plurality of operators depending on the contents of the medical orders. Thus, authorization to operate one medical apparatus 10 may be given to either one operator or a plurality of operators. Also, authorization to operate a plurality of medical apparatuses 10 may be given to either one operator or a plurality of operators. Specifically, if one examination is performed with a plurality of medical apparatuses 10, as in the case of angio-CT examination, the medical apparatus information includes a plurality of medical apparatuses 10 with respect to one medical order ID, and the operator information includes a plurality of operators.

The scheduled date and time are information that identifies the date and time when the order is to be executed. The patient information is information that identifies a patient which is a subject of a medical act, and includes, for example, a patient ID that unambiguously identifies the patient, and the name, gender, birthday, and blood type of the patient. The patient information may include information that identifies physical characteristics, such as a body height, a body weight, and the like, or a preexisting disorder. The order information includes, for example, contents of the order, such as an examination, medical treatment, image interpretation, clinical examination, and the like.

The processing circuitry 33 is realized by, for example, a processor such as a central processing unit (CPU), a graphics processing unit (GPU), or the like. The processing circuitry 33 controls each of the units of the medical information processing apparatus 30, thereby controlling the whole of the medical information processing apparatus 30. The processing circuitry 33 reads and executes programs stored in the memory 31, thereby functioning as an operator information acquisition section 33a, an apparatus information acquisition section 33b, and a provision section 33c.

The operation apparatus 20 performs communications with the medical apparatus 10 or the medical information processing apparatus 30. The operation apparatus 20 comprises, for example, a terminal, such as a personal computer (PC). The operators of the operation apparatus 20 are, for example, medical staff such as a primary doctor, an image interpretation doctor, a radiation technologist, a clinical laboratory technologist, a nurse, and the like. The operation apparatuses 20a-20n may be disposed in the same room or different rooms in the same medical facility, or may be disposed in different medical facilities. The operation apparatus 20 may be disposed in the same room as or a different room from the room where the medical apparatus 10 is disposed (examination room). Examples of the different room include, for example, a room adjacent to the examination room where the medical apparatus 10 is disposed and the operator can directly observe the medical apparatus 10, and a room far away from the place where the medical apparatus 10 is disposed and monitored by the operator via the common network.

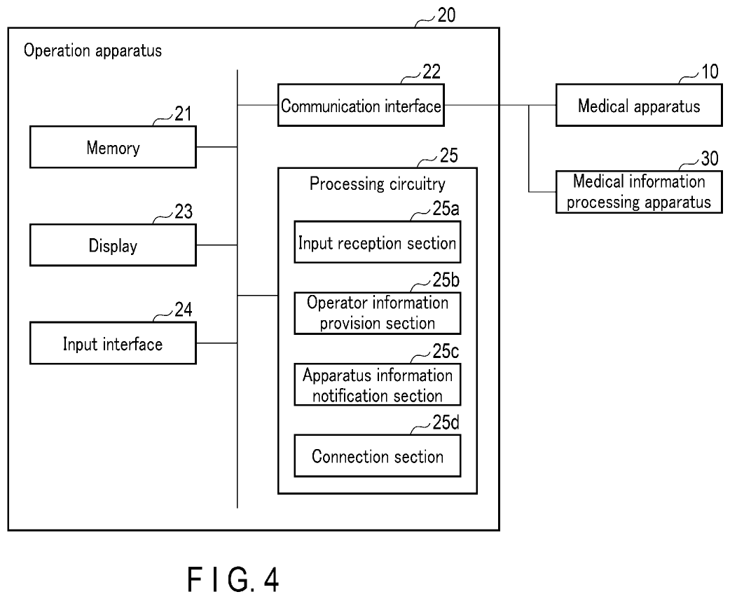

As shown in FIG. 4, the operation apparatus 20 includes a memory 21, a communication interface 22, a display 23, an input interface 24, and processing circuitry 25.

The memory 21 is realized by, for example, a semiconductor memory device such as a RAM or a flash memory, or a hard disk, an optical disk, etc. The memory 21 stores various data and programs necessary for processing performed by the processing circuitry 25 and various data obtained through processing performed by the processing circuitry 25. The memory 21 stores various information for use in operation of the medical apparatus 10, a list of medical information acquired from the medical information processing apparatus 30, and the like. The memory 21 is an example of a storage.

The communication interface 22 is realized by, for example, a communication apparatus, such as a network card, a network adapter, an NIC, or the like. The communication interface 22 performs communications with the medical apparatuses 10 or the medical information processing apparatus 30 via the network.

The display 23 is realized by, for example, a display device such as a liquid crystal display, a cathode ray tube (CRT) display, or the like. The display 23 displays medical information generated by the processing circuitry 25, a graphical user interface (GUI) for receiving various operations from an operator, and the like.

The display 23 also displays a list of the medical apparatuses 10 operable by the operator. The display 23 is an example of a display unit.

The input interface 24 receives various types of input operations from the operator, converts the received input operation into an electric signal, and outputs the electric signal to the processing circuitry 25. For example, the input interface 24 receives inputs of medical information, inputs of various types of command signals, or the like from the operator. The input interface 24 is realized by a mouse or a keyboard, a trackball, a switch button, a touch screen in which a display screen and a touch pad are integrated, a non-contact input circuit using an optical sensor, a voice input circuit, or the like, for performing various processing of the processing circuitry 25. The input interface 24 is connected to the processing circuitry 25 and adapted to convert input operations received from the operator into electric signals and to output the electric signals to the control circuitry. The input interface is not limited to a device having physical operational components, such as a mouse, a keyboard, or the like. That is, examples of the input interface also include processing circuitry for electric signals, which is adapted to receive an electric signal corresponding to an input operation from an external input device separate from the apparatus, and to output this electric signal to the processing circuitry 25. The input interface 24 is an example of an input unit.

The processing circuitry 25 is realized by, for example, a processor, such as a CPU, a GPU, or the like. The processing circuitry 25 controls the entire operation of the operation apparatus 20. In addition, the processing circuitry 25 causes the display 23 to display various types of information. For example, the processing circuitry 25 causes the display 23 to display a login screen to authenticate the operator, a list of medical orders, and various operation screens to perform operations of the medical apparatus 10. In addition, the processing circuitry 25 reads and executes programs stored in the memory 21, thereby functioning as an input reception section 25a, an operator information provision section 25b, an apparatus information notification section 25c, and a connection section 25d.

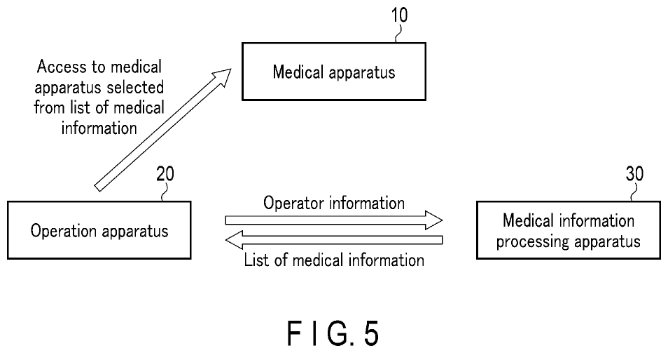

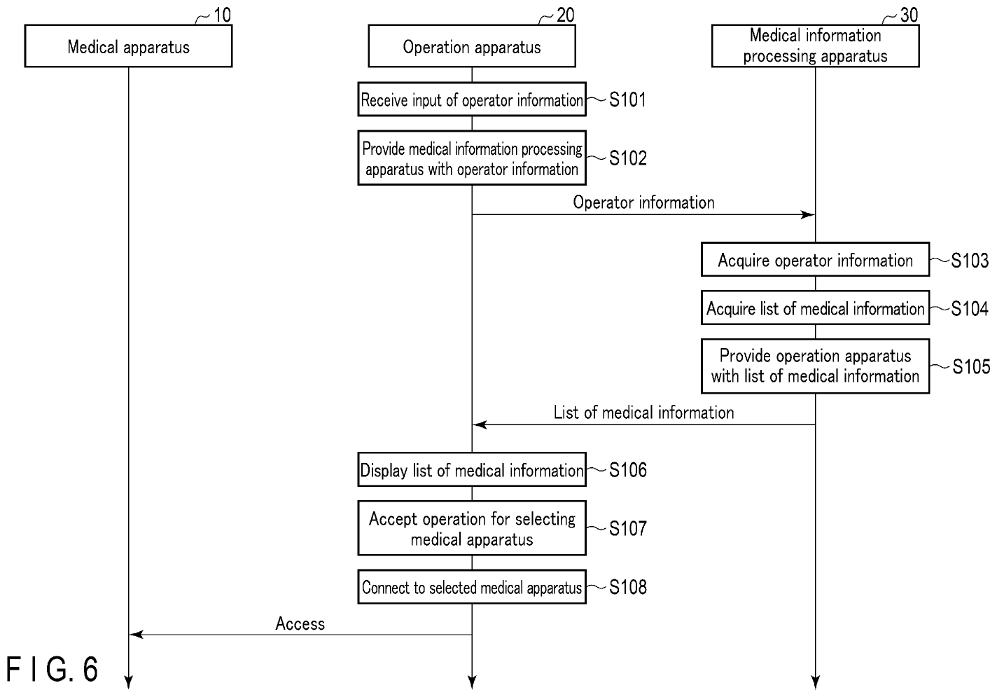

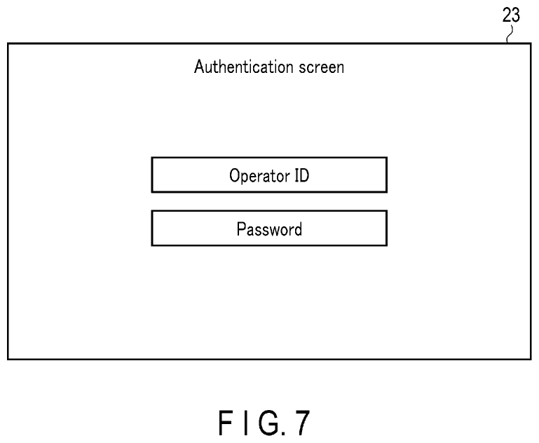

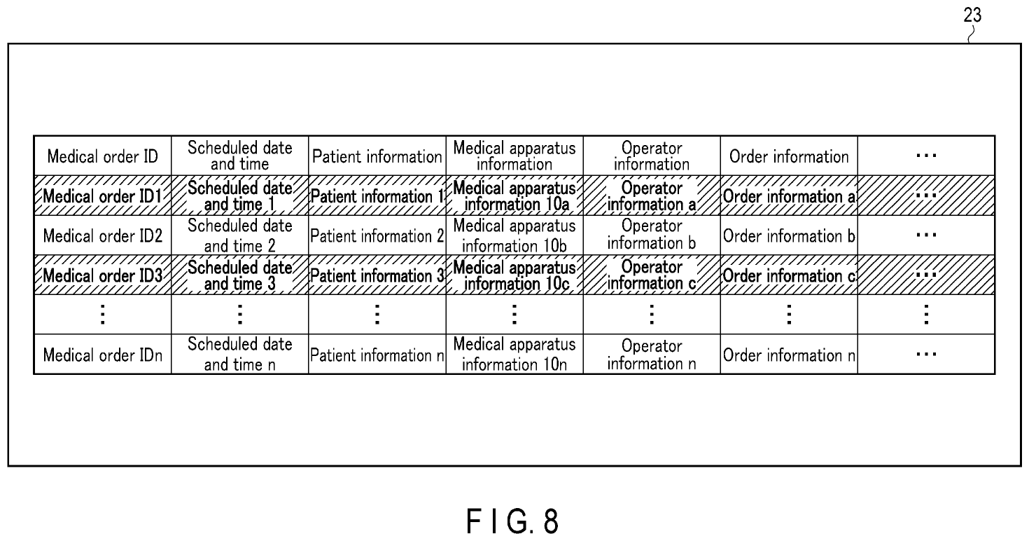

Operations of the medical information processing system 1 configured as described above will be explained with reference to FIGS. 5 to 8. FIG. 5 is a diagram showing communication processing between structural elements of the medical information processing system 1. FIG. 6 is a sequence diagram for explaining operations of the medical information processing system 1. FIG. 7 is a diagram showing an example of a display screen of authentication information. FIG. 8 is a diagram showing an example of a display screen of apparatus information.

The input reception section 25a of the operation apparatus 20 displays an authentication screen shown in FIG. 7 on the display 23, and receives input of operator information that identifies the operator (an operator ID, a password, an operator name, and the like) (step S101). The operator information provision section 25b provides the medical information processing apparatus 30 with the operator information received by the input reception section 25a via the common network (step S102).

The operator information acquisition section 33a of the medical information processing apparatus 30 acquires the operator information provided from the operation apparatus 20 (step S103). The apparatus information acquisition section 33b acquires apparatus information indicating the medical apparatus 10 operable by the operator of the operation apparatus 20 based on the operator information acquired by the operator information acquisition section 33a. Specifically, upon acquisition of the operator information, the apparatus information acquisition section 33b acquires the list of medical information stored in the memory 31 as apparatus information (step S104).

The provision section 33c provides the apparatus information acquired from the apparatus information acquisition section 33b in such a form that the medical apparatuses 10 operable by the operator of the operation apparatus 20 can be identified from the other medical apparatuses 10. For example, the provision section 33c extracts the medical information including the operator information acquired in step S103 from the apparatus information (the list of medical information). The provision section 33c converts the extracted medical information in such a form as to identify the medical apparatuses operable by the operator.

The provision section 33c also provides the apparatus information (the list of medical information) acquired from the apparatus information acquisition section 33b in such a form as to be connectable to the medical apparatus 10 operable by the operator of the operation apparatus 20. For example, if connection information (link information) to connect to the medical apparatus 10, such as the URL of the medical apparatus 10, is included (provided) in each medical information in the list of medical information acquired by the apparatus information acquisition section 33b, the provision section 33c deletes the connection information included in the medical information other than the medical information extracted from the list of medical information.

On the other hand, if connection information (link information) to connect to the medical apparatus 10 is not included in each piece of medical information in the list of medical information acquired by the apparatus information acquisition section 33b, the provision section 33c adds the connection information only to the medical information extracted from the list of medical information. As a result, the connection information is added only to the medical apparatus 10 operable by the operator of the operation apparatus 20. The provision section 33c provides the operation apparatus 20, which is a transmission source of the operator information, with the apparatus information (the list of medical information) that has been subjected to the various processing described above via the common network (step S105).

The apparatus information notification section 25c of the operation apparatus 20 provides notification of the apparatus information (the list of medical information) provided from the medical information processing apparatus 30. Specifically, the apparatus information notification section 25c causes the display 23 to display the apparatus information provided from the medical information processing apparatus 30 (step S106). The display 23 displays, for example, the apparatus information shown in FIG. 8. The apparatus information is displayed in a form in which the medical apparatuses 10 operable by the operator of the operation apparatus 20 can be identified from the medical apparatuses 10 not operable by the operator. More specifically, the medical information including the medical apparatuses 10 operable by the operator of the operation apparatus 20 is emphasized with highlighting or bold face. As a result, the operator of the operation apparatus 20 can understand the medical order that he or she is going to execute and can identify the medical apparatuses 10 operable by himself/herself from the other medical apparatuses 10.

The connection section 25d accepts an operation for selecting a medical apparatus 10 (step S107). For example, the operator selects the medical order (for example, a medical order ID1) he or she is going to execute from the apparatus information (the list of medical information) displayed on the display 23 through an operation via the input interface 24. As a result, the connection section 25d recognizes that the medical apparatus 10 corresponding to the medical order selected by the operator (the medical apparatus 10 represented by the medical apparatus information 10a) is going to be operated by the operator.

The connection section 25d performs processing for connecting to the selected medical apparatus 10 (step S108). Specifically, the connection section 25d connects to the medical apparatus 10 selected by the operator based on the connection information (link information) included in the medical information corresponding to the selected medical order (step S108).

When the operation apparatus 20 is connected to the medical apparatus 10, an operation screen for operating the selected medical apparatus 10 is displayed on the display 23 of the operation apparatus 20. The operation apparatus 20 performs a medical act corresponding to the medical order in accordance with the operation of the operator via the input interface 24.

As described above, according to the present embodiment, the operation apparatus 20 can be provided with the apparatus information including the medical apparatus operable by the operator of the operation apparatus 20 (the list of medical information) from the medical information processing apparatus 30. The operator of the operation apparatus 20 can confirm the medical apparatus 10 operable by himself/herself by confirming the apparatus information provided from the medical information processing apparatus 30. Therefore, a medical apparatus 10 that should not be operated by the operator of the operation apparatus 20 can be prevented from being erroneously operated.

Furthermore, the medical information processing apparatus 30 provides the operation apparatus 20 with apparatus information in such a form that the operator of the operation apparatus 20 can identify the medical apparatus 10 operable by himself/herself. As a result, a medical apparatus 10 that should not be operated by the operator of the operation apparatus 20 can be prevented from being erroneously operated.

In addition, the medical information processing apparatus 30 provides the apparatus information to be provided for the operation apparatus 20 in such a form that the operation apparatus 20 can be connected to the medical apparatuses 10 operable by the operator of the operation apparatus 20. Therefore, the operator of the medical apparatus 20 can easily access the medical apparatus 10 that should be operated by himself/herself, and an operation apparatus 10 that should not be operated by the operator of the operation apparatus 20 can be prevented from being erroneously operated.

### (Modification of First Embodiment)

In the embodiment described above, a case is described in which information on the medical apparatus 10 operable by the operator of the operation apparatus 20 is provided in such a form that the operation apparatus 20 can be connected to the medical apparatus 10. However, the information need not be provided in such a connectable form. For example, the operation apparatus 20 may search a plurality of medical apparatuses 10a to 10n for a medical apparatus 10 operable by the operator, and may be connected to the corresponding medical apparatus 10.

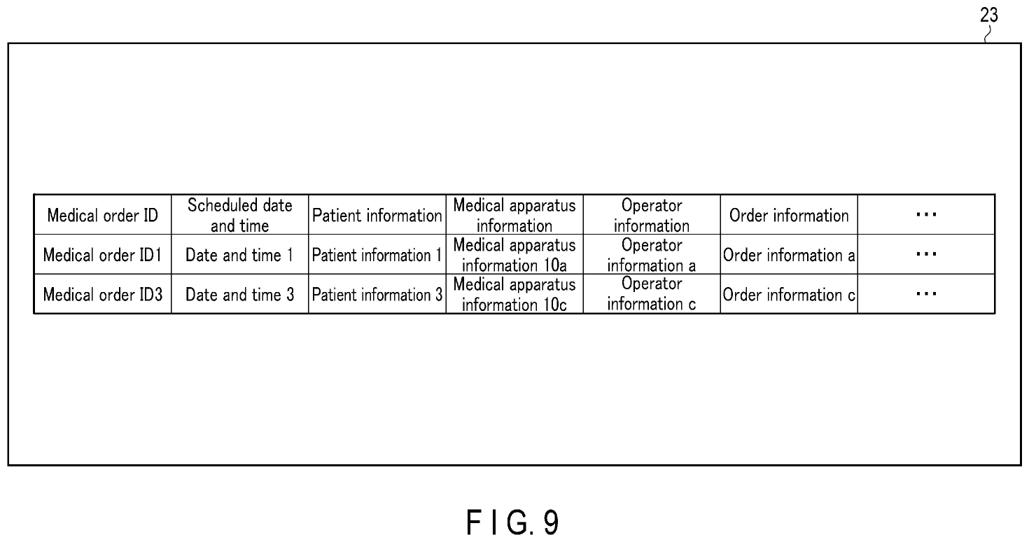

Furthermore, in the embodiment described above, a case is described in which information is provided in such a form that the operator of the operation apparatus 20 can identify the medical apparatus 10 operable by himself/herself from the other medical apparatuses 10. However, the information need not be provided in such an identifiable form. For example, the apparatus information acquisition section 33b of the medical information processing apparatus 30 may read, from the memory 31, apparatus information including operator information (of the same operator) provided from the operation apparatus 20, and the provision section 33c may provide the operation apparatus 20 with the apparatus information (the list of medical information). In this case, the display 23 of the operation apparatus 20 displays the apparatus information (the list of medical information) indicating the medical apparatus 10 operable by the operator of the operation apparatus 20, as shown in FIG. 9. As a result, a medical apparatus 10 that should not be operated by the operator of the operation apparatus 20 can be prevented from being erroneously operated.

Furthermore, in the embodiment described above, a case is described in which operator information (an operator) and medical apparatus information (a medical apparatus 10) are associated for each medical order in the medical information processing apparatus 30. However, the operator information and the medical apparatus information need not be associated for each medical order. For example, the medical information processing apparatus 30 may store the operator information and the medical apparatus information in association with each other, and may provide the list of medical information (apparatus information) including the medical apparatus information associated with the operator information received from the operation apparatus 20.

Furthermore, in the embodiment described above, a case is described in which the medical information processing apparatus 30 stores, for each medical order, the medical information indicating the medical apparatus 10 operable by the operator. However, the medical information processing apparatus 30 may store a workflow of medical processing determined in accordance with the medical order (a series of medical processing comprising one or more steps) as medical information. In this case, the operator information acquisition section 33a acquires the operator information provided from the operation apparatus 20, and the apparatus information acquisition section 33b acquires a workflow including the operator information acquired by the operator information acquisition section 33a. The workflow includes apparatus information indicating the medical apparatus 10 operable by the operator. The provision section 33c provides the operation apparatus 20 (which is a transmission source of the operator information) with the workflow including apparatus information acquired by the apparatus information acquisition section 33b.

### Second Embodiment

Next, the second embodiment will be described. In the second embodiment, apparatus information in consideration of a state of use of the medical apparatus 10 is provided.

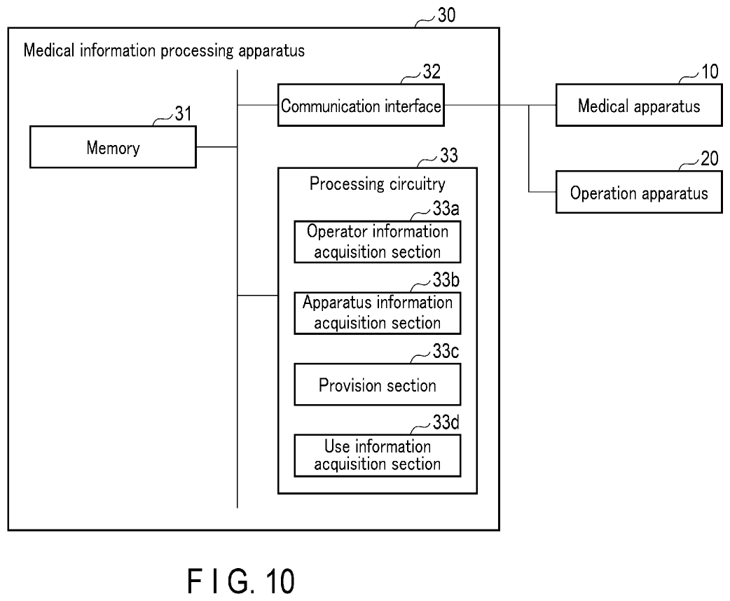

FIG. 10 is a diagram showing a configuration of the medical information processing apparatus 30 according to the present embodiment. The medical information stored in the memory 31 includes use information in addition to the various kinds of information shown in FIG. 3. The use information includes information indicating a state of use of the medical apparatus 10. Specifically, examples of the state of use of the medical apparatus 10 include a progress rate of the medical order, a time to wait until the medical apparatus 10 is ready for use, an operator who is operating the medical apparatus 10, information on the operation apparatus 20 which the operator is using, and the like. The use information is transmitted from each medical apparatus 10 to the medical information processing apparatus 30 at regular time intervals.

The processing circuitry 33 reads and executes programs stored in the memory 31, thereby functioning as an operator information acquisition section 33a, an apparatus information acquisition section 33b, a provision section 33c, and a use information acquisition section 33d.

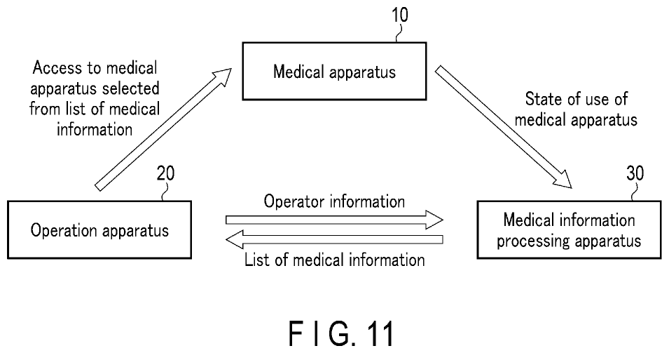

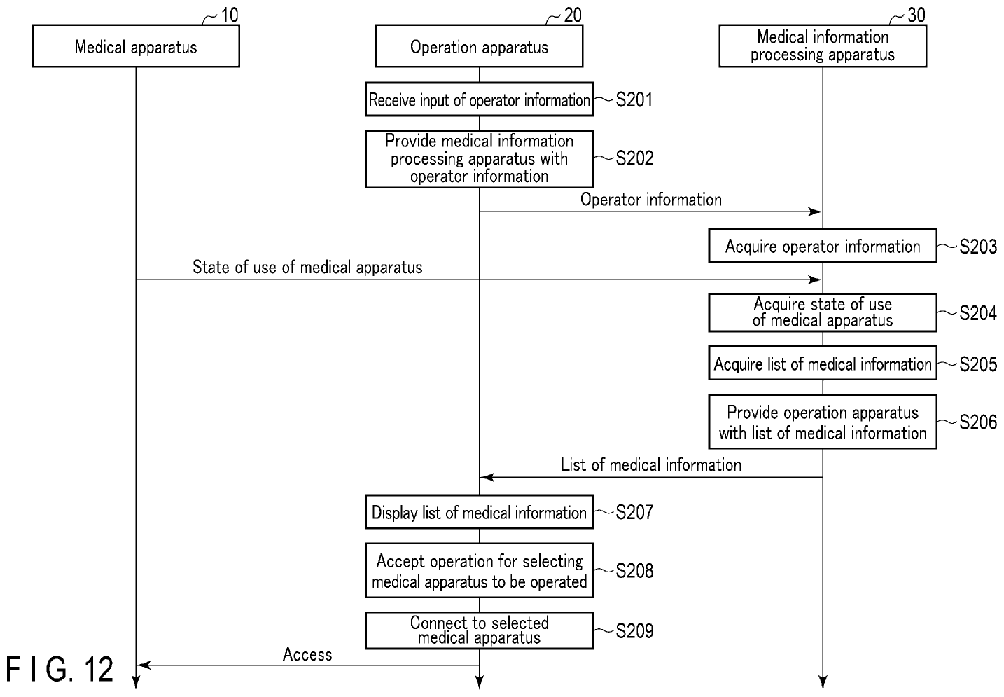

The use information acquisition section 33d, as shown in FIGS. 11 and 12, acquires the use information from each medical apparatus 10 via the network (step S204), and includes the acquired use information in the corresponding medical information in the memory 31. Specifically, the use information acquisition section 33d updates the use information associated with the medical apparatus 10 which is a transmission source of the use information to the acquired use information in the medical information list.

The provision section 33c provides the operation apparatus 20 with the list of medical information including the medical apparatus information associated with the operator information (apparatus information) received from the operation apparatus 20 (step S206). The medical information includes the use information indicating a latest state of use of the medical apparatus 10.

The medical information processing apparatus 30 may repeat the processing from step S205 to step S208 each time the state of use is acquired from the medical apparatus 10 and new use information is acquired at predetermined time intervals. As a result, the operation apparatus 20 is provided with the apparatus information reflecting the latest state of use.

According to the present embodiment, the medical information processing apparatus 30 can provide the operation apparatus 20 operated by the operator with apparatus information including use information indicating the state of use of the medical apparatus 10. Therefore, a medical apparatus 10 that should not be operated by the operator of the operation apparatus 20 can be prevented from being erroneously operated. In addition, the operator of the operation apparatus 20 can operate the medical apparatus 10 in consideration of the state of use of the medical apparatus 10.

### (Modification of Second Embodiment)

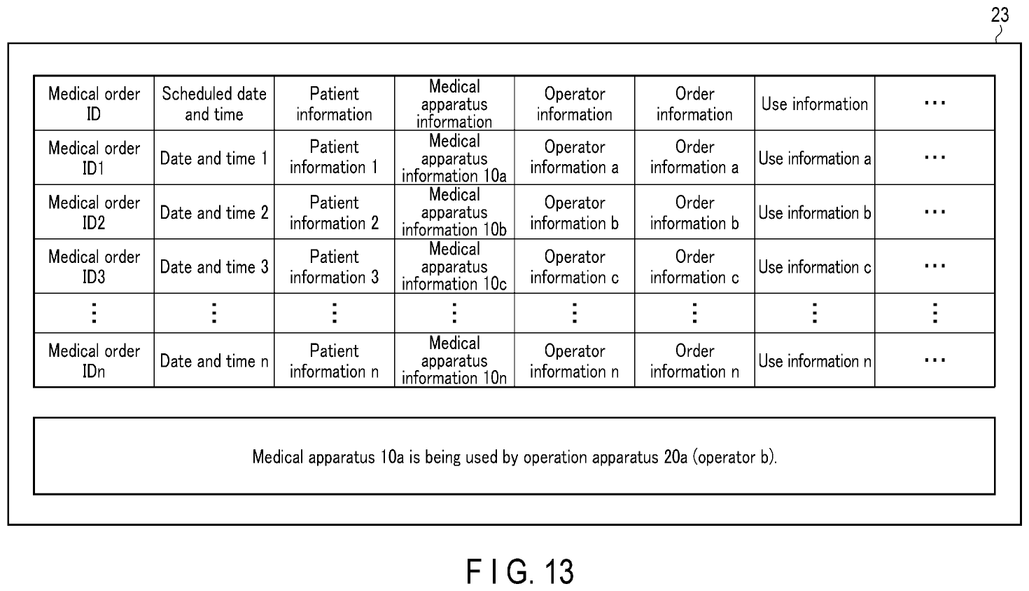

The medical information processing apparatus 30 may provide the information in such a form that the operator of the operation apparatus 20 can identify the medical apparatus 10 operable by himself/herself based on the use information. Accordingly, depending on the state of use of the medical apparatus 10, even if the operator has authorization to operate the medical apparatus 10, a medical apparatus 10 that should not be operated by the operator can be prevented from being erroneously operated. In this case, for example, as shown in FIG. 13, the operator may be informed that the medical apparatus 10 is being used for another medical order. At this time, the operation apparatus 20 that is presently operating the selected medical apparatus 10 and the operator thereof may also be notified together. The medical information processing apparatus 30 may give priority to the medical apparatuses 10 operable by the operator of the operation apparatus 20 based on the use information (the state of use of the medical apparatuses 10). Accordingly, even if the operator has authorization to operate the medical apparatuses 10, the operator can select a medical apparatus 10 depending on the state of use of the medical apparatuses 10. The medical information processing apparatus 30 may provide information in such a form that the operator of the operation apparatus 20 can connect to the medical apparatus 10 operable by himself/herself based on the use information. Accordingly, even if the operator has authorization to operate the medical apparatus 10, a medical apparatus 10 that should not be operated by the operator in accordance with the state of use of the medical apparatus 10 can be prevented from being erroneously operated.

In the embodiment described above, a case is described in which the medical apparatus 10 provides the medical information processing apparatus 30 with a report of the state of use of the medical apparatus 10. However, the state of use of the medical apparatus 10 may be reported to the operation apparatus 20 that operates the medical apparatus 10.

### Third Embodiment

Next, the third embodiment will be described. In the third embodiment, a case is described in which when the medical apparatus 10 is accessed from the operation apparatus 20, the medical apparatus 10 requests authentication information from the operator.

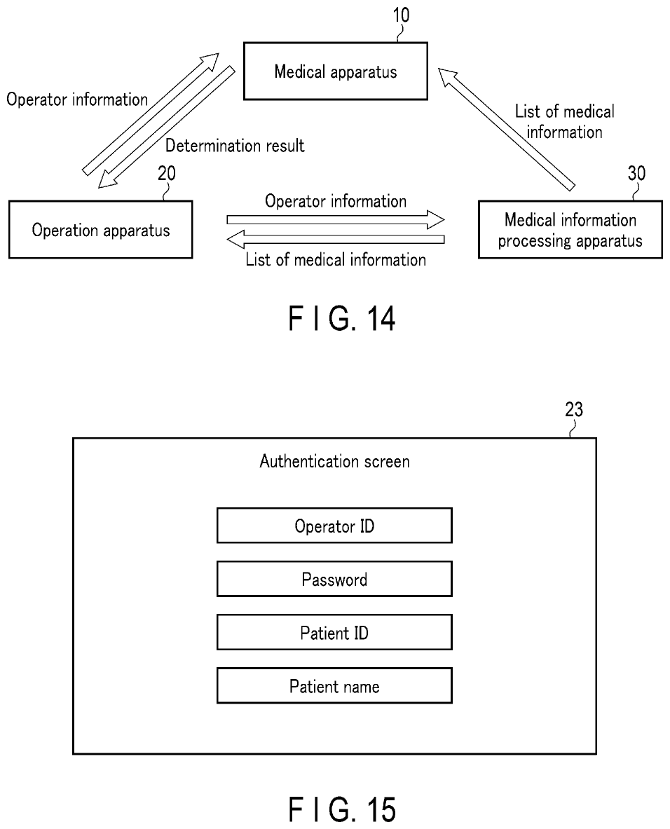

FIG. 14 is a diagram showing communication processing between structural elements of the medical information processing system 1 of this embodiment. The memory of each medical apparatus 10 stores a list of medical information in which an operator is associated with a medical apparatus operable by the operator. The list of medical information is periodically provided, for example, via the network from the medical information processing apparatus 30 to each medical apparatus 10.

In the same manner as in the first embodiment, the list of medical information is provided from the medical information processing apparatus 30 to the operation apparatus 20, and the list of the medical information is displayed on the operation apparatus 20. Then, the operator of the operation apparatus 20 performs an operation for selecting a medical apparatus 10 which he/she is going to operate from the plurality of displayed medical apparatuses connected to the network. The operation apparatus 20 performs communications with the medical apparatus 10 selected by the operator. The medical apparatus 10 requests operator information in response to an access from the operation apparatus 20. The operation apparatus 20 causes the display 23 to display an input screen for the operator information in response to the request from the medical apparatus 10. FIG. 15 is a diagram showing an example of an input screen of the operator information. The input screen for the operator information indicates boxes in which, for example, an operator ID, a password, a patient ID, and a patient name are to be input. Upon input of information through the input screen for the operator information, the operator information provision section 25b of the operation apparatus 20 transmits the input information to the medical apparatus 10 via the network.

The medical apparatus 10 identifies the operator who is operating the operation apparatus 20 using the information transmitted from the operation apparatus 20 and the list of medical information stored in the memory, determines whether the identified operator is associated with the medical apparatus 10, and transmits the determination result to the operation apparatus 20 via the network.

If the identified operator is not associated with the medical apparatus 10 in the list of medical information, the medical apparatus 10 determines that the accessing operator is not an operator who is allowed to operate the medical apparatus 10, and does not accept the operation by the operator. On the other hand, if the identified operator is associated with the medical apparatus 10 in the list of medical information, the medical apparatus 10 determines that the accessing operator is an operator who is allowed to operate the medical apparatus 10, and accepts the operation by the operator.

In the present embodiment, the medical apparatus 10 stores the list of medical information. When the operator accesses the medical apparatus 10, the accessed medical apparatus 10 authenticates whether the accessing operator is allowed to operate the medical apparatus 10, so that an unintended use of the medical apparatus 10 by the operator can be effectively suppressed.

Furthermore, in a state where an operator is accessing a medical apparatus 10 from one operation apparatus 20, if the same operator accesses the same medical apparatus 10 from another operation apparatus 20, the accessed medical apparatus 10 may accept only an operation from the operation apparatus 20 that was accessed lastly. For example, in a state where an operator is accessing a medical apparatus 10, if the operator intends to access and operate the medical apparatus 10 from another operation apparatus 20b, the medical apparatus 10 accepts only an operation from the operation apparatus 20b that was accessed lastly, but does not accept an operation from the operation apparatus 20 that the operator logged into previously. With this configuration, for example, if the operation apparatus 20 that is operating after starting a medical order has a problem and the operator accesses the medical apparatus 10 through another operation apparatus 20 to continue operation, the operation from the operation apparatus 20 having the problem cannot be accepted. Therefore, a malfunction due to problems with the operation apparatus 20 can be prevented.

### Fourth Embodiment

Next, the fourth embodiment will be described. In the fourth embodiment, results of medical orders are sequentially reported to a medical information processing apparatus 30 from medical apparatuses 10.

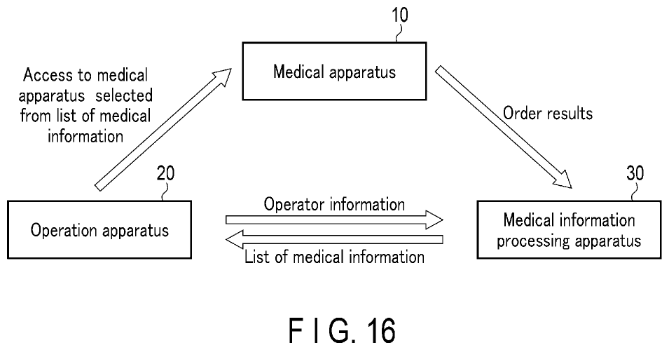

FIG. 16 is a diagram showing communication processing between structural elements of a medical information processing system 1. In this embodiment, the medical information processing apparatus 30 acquires results of the medical orders executed by using the medical apparatuses 10 (hereinafter referred to as the order results) via the common network from the respective medical apparatuses 10. Examples of the order results include, for example, image data generated by a medical image diagnostic apparatus, examination data, and memory address information indicating the location (destination to save) of the examination data. The order results are transmitted from each medical apparatus 10 to the medical information processing apparatus 30 at regular time intervals.

The medical information processing apparatus 30 stores the acquired order results in a memory 31. At this time, the acquired order results may be stored in association with the list of medical orders, or may be stored as independent information. However, even if the order results are stored as independent information, it is preferable that identification information (for example, a medical order ID) to be associated with the list of medical orders be added to the order results.

The order results may be generated by each of the operation apparatuses 20. In this case, the order results are transmitted from each operation apparatus 20 to the medical information processing apparatus 30.

According to at least one of the embodiments described above, in the medical information processing system in which one or more medical apparatuses and one or more operation apparatuses are connected via a common network, a medical apparatus that should not be operated by an operator can be prevented from being erroneously operated.

In the description above, the medical information processing apparatus 30 is an apparatus independent of the medical apparatus 10 or the operation apparatus 20. However, the medical apparatus 10 or the operation apparatus 20 may include each of the configurations of the medical information processing apparatus 30. For example, the functions executed by the processing circuitry 33 of the medical information processing apparatus 30 may be executed by the processing circuitry of each medical apparatus 10 or the processing circuitry 25 of each operation apparatus 20. In this case, the medical information stored in the memory 31 of the medical information processing apparatus 30 is stored in advance in the memory of each medical apparatus 10 or the memory 21 of each operation apparatus 20.

While the sections shown in FIGS. 2, 4, and 10 are described as being realized by the single processing circuitry 25 and 33, the processing circuitry may be constituted by a combination of a plurality of independent processors respectively running programs to realize the functions. Alternatively, the sections of the processing circuitry 25 and 33 may be implemented as separate hardware circuits.

Each of the operation apparatus 20 and the medical information processing apparatus 30 is described above as performing a plurality of functions with a single console; however, a plurality of functions may be executed by separate apparatuses. For example, the sections of the processing circuitry 25 and 33 may be implemented on different apparatuses in a distributed manner.

The term “processor” used in the above description refers to, for example, circuitry such as a central processing unit (CPU), a graphics processing unit (GPU), an application specific integrated circuit (ASIC), a programmable logic device such as a simple programmable logic device (SPLD) or a complex programmable logic device (CPLD), a field programmable gate array (FPGA), etc. If the processor is, for example, a CPU, the processor realizes its function by reading and executing the program stored in the storage circuitry. On the other hand, if the processor is, for example, an ASIC, the function corresponding to a program is directly incorporated into a circuit of the processor as a logic circuit, instead of being stored in the storage circuitry. The processors described in connection with the above embodiments are not limited to single-circuit processors; a plurality of independent circuits may be integrated into a single processor that realizes the functions. In addition, a plurality of structural elements may be integrated into one processor to realize the functions of the structural elements. The above description of the “processor” is applicable to the subsequent embodiments and modifications.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry,
wherein the processing circuitry is configured to:
acquire operator information that identifies an operator of an operation apparatus;
acquire apparatus information that indicates a medical apparatus operable by the operator based on the acquired operator information; and
provide the operation apparatus with the acquired apparatus information.

2. The medical information processing apparatus according to claim 1, further comprising a storage that stores a plurality of pieces of medical information in which the operator and the medical apparatus operable by the operator are associated,
wherein the processing circuitry is configured to acquire apparatus information that indicates the medical apparatus associated with the operator identified by the operator information from among the plurality of pieces of medical information stored in the storage.

3. The medical information processing apparatus according to claim 1, further comprising a storage that stores a plurality of pieces of medical information in which the operator and the medical apparatus operable by the operator are associated,
wherein the processing circuitry is configured to acquire, as the apparatus information, the pieces of medical information stored in the storage in response to acquisition of the operator information.

4. The medical information processing apparatus according to claim 3, wherein the processing circuitry is configured to provide the acquired apparatus information in a form such that the medical apparatus operable by the operator is identifiable from other medical apparatuses.

5. The medical information processing apparatus according to claim 1, wherein the processing circuitry is configured to provide the acquired apparatus information in a form of being connectable to the medical apparatus operable by the operator.

6. The medical information processing apparatus according to claim 1, wherein the processing circuitry is configured to:
acquire use information that indicates a state of use of the medical apparatus; and
acquire apparatus information that indicates the medical apparatus operable by the operator based on the operator information and the use information.

7. The medical information processing apparatus according to claim 1, wherein the processing circuitry is configured to:
acquire use information that indicates a state of use of the medical apparatus; and
provide the acquired apparatus information in a form such that the state of use of the medical apparatus operable by the operator is identifiable.

8. A medical information processing system comprising an operation apparatus and a medical information processing apparatus connected to the operation apparatus via a network,
the operation apparatus comprising processing circuitry configured to receive an input of operator information that identifies an operator, and to provide the medical information processing apparatus with the operator information,
the medical information processing apparatus comprising processing circuitry configured to acquire the operator information provided from the operation apparatus, to acquire apparatus information that indicates a medical apparatus operable by the operator based on the acquired operator information, and to provide the operation apparatus with the acquired apparatus information.

9. The medical information processing system according to claim 8, wherein the processing circuitry of the operation apparatus is configured to report the apparatus information provided from the medical information processing apparatus.

10. The medical information processing system according to claim 9, further comprising a medical apparatus connected to the operation apparatus via the network,
wherein the processing circuitry of the operation apparatus is configured to accept an operation for selecting the medical apparatus to be operated by the operator from the apparatus information, and to connect to the medical apparatus selected by the operation via the network.
